(19) United States
(12) Patent Application Publication
(10) Pub. No.: US 2026/0060519 A1
(43) Pub. Date: Mar. 5, 2026

(54) IMAGE SENSOR DEVICE AND METHOD FOR IMAGE AND LIGHT SOURCE SYNCHRONIZATION THEREOF, ENDOSCOPIC DEVICE

(71) Applicant: HUKUI BIOTECHNOLOGY CO., LTD., Taipei City (TW)

(72) Inventors: SHIH-CHE LO, Taipei City (TW); YI-MING CHU, Taipei City (TW)

(73) Assignee: HUKUI BIOTECHNOLOGY CO., LTD., Taipei City (TW)

(21) Appl. No.: 19/221,536

(22) Filed: May 29, 2025

Related U.S. Application Data
(60) Provisional application No. 63/690,813, filed on Sep. 5, 2024.

(30) Foreign Application Priority Data
Feb. 27, 2025 (TW) 114107547

(51) Int. Cl. A61B 1/06; A61B 1/00; A61B 1/04; A61B 1/045; A61B 1/05; A61B 1/07; G16H 40/63

(52) U.S. Cl. CPC A61B 1/0655; A61B 1/0002; A61B 1/00124; A61B 1/00186; A61B 1/043; A61B 1/045; A61B 1/046; A61B 1/05; A61B 1/063; A61B 1/0638; A61B 1/0661; A61B 1/07; G16H 40/63; A61B 2560/0462

(57) ABSTRACT

An image sensor device, which is suitable for coupling with an image processor and a light source device which used to emit at least two kinds of light sources. The image sensor device includes an image sensor and a timing control module. The image sensor is used to sense reflected light generated by a part to be measured when illuminated by the light source device, and convert the light signal corresponding to the reflected light into an image signal and then transmit it to the image processor. The timing control module uses a synchronization signal to synchronously control the light source device and the image sensor. According to the different light sources required by the image sensor in each frame, the light source device is synchronously controlled to switch and activate between the light sources, so that the image sensor generates different image signals corresponding to different light sources.

### CROSS-REFERENCE TO RELATED APPLICATION

This non-provisional application claims priority under 35 U.S.C. § 119 (a) on U.S. provisional Patent Application No. 63/690,813 filed on Sep. 5, 2024, and on patent application No. 114107547 filed in Taiwan, R.O.C. on Feb. 27, 2025, the entire contents of which are hereby incorporated by reference.

### BACKGROUND OF THE INVENTION

### 1. Field of the Invention

The present disclosure relates to an image sensor device, and particularly relates to an image sensor device for synchronous operation of an image and a light source, a method for image and light source synchronization thereof, and an endoscope device provided with the image sensor device.

### 2. Description of the Related Art

The incidence of esophageal cancer and head and neck cancer is increasing year by year, and when symptoms occur and a diagnosis is made, a physician will first perform endoscopy with a white light source, and then switches the white light source to a narrow band imaging light source for reconfirmation when a suspected symptom is found, so that at each moment, only imaging of one kind of light source is available for the physician to interpret.

### BRIEF SUMMARY OF THE INVENTION

When a physician performs endoscopy, an endoscope may displace during the operation, consequently, relative position of images shot under different light sources may be different, and a narrow band imaging light image corresponding to a white light image cannot be accurately obtained for the location of a suspected symptom, and as a result, the physician cannot perform accurate judgment on the symptom.

The applicant devoted to researching and developed an image sensor device and a method for image and light source synchronization thereof, which could achieve the purpose of synchronous operation on the image and the light source.

A second objective of the present disclosure is to provide an image sensor device and a method for image and light source synchronization thereof, and a plurality of image pictures generated by switching different light sources may look like continuous pictures in human eyes.

The present disclosure provides an image sensor device, which is suitable for coupling with an image processor and a light source device. The light source device is configured to emit at least two kinds of light sources to illuminate a part to be measured. The image sensor device includes an image sensor and a timing control module. The image sensor is electrically connected to the image processor and is configured to sense reflected light generated by the part to be measured when illuminated by the light source device, and convert a light signal corresponding to the reflected light into an image signal and then transmit the image signal to the image processor. The timing control module is electrically connected to the image sensor and is configured to use a synchronization signal to synchronously control the light source device and the image sensor, and according to different light sources required by the image sensor in each frame, synchronously control the light source device to switch and activate between the light sources, so that the image sensor generates different image signals corresponding to different light sources.

In an embodiment, the light sources emitted by the light source device are at least two of white light, structured light, infrared light, ultraviolet light, fluorescence excitation light and narrow band imaging light.

In an embodiment, the timing control module integrates a register for storing an operation code, and the operation code is configured for setting respective clock cycles of the light sources emitted by the light source device.

In an embodiment, the timing control module integrating the register is further externally equipped with or integrates an external memory. The external memory is electrically connected to the register, and the register reads the external memory to obtain the operation code.

In an embodiment, the image sensor transmits the image signal and the respective clock cycles of the light sources to the image processor by means of a transmission port, and the transmission port is a mobile industry processor interface.

In an embodiment, the image sensor sets a corresponding exposure parameter according to different light sources required in each frame.

In an embodiment, the exposure parameter includes a shutter time and a gain value.

In an embodiment, the image sensor device according to the present disclosure further includes an optical filter, which is arranged in front of the image sensor to filter out light rays with certain wavelengths.

The present disclosure further provides an endoscope device, which is suitable for combining with a light source device. The endoscope device includes an extension tube, an imaging assembly and a light guide member. The extension tube is provided with a proximal end and a distal end opposite to the proximal end. The imaging assembly includes a lens group located close to the distal end of the extension tube, and the image sensor device positioned in the extension tube and clamped to the lens group. The light guide member is located close to the distal end of the extension tube and is beside the lens group. The light source device is suitable for being located in the extension tube and close to the light guide member.

In an embodiment, the endoscope device according to the present disclosure further includes an optical filter arranged in front of the image sensor device.

The present disclosure further provides a method for image and light source synchronization of an image sensor device, which includes the following steps: providing a light source device for emitting at least two kinds of light sources; providing an image sensor device which includes an image sensor and a timing control module, where the timing control module is electrically connected to the image sensor; transmitting a synchronization signal to the timing control module; and synchronously controlling the light source device and the image sensor by the timing control module using the synchronization signal, and according to different light sources required by the image sensor in each frame, synchronously controlling the light source device to switch and activate between the light sources, so that the image sensor generates different image signals corresponding to different light sources.

In an embodiment, the light sources emitted by the light source device are at least two of white light, structured light, infrared light, ultraviolet light, fluorescence excitation light and narrow band imaging light.

In an embodiment, the timing control module integrates a register for storing an operation code, and the operation code is configured for setting respective clock cycles of the light sources emitted by the light source device.

In an embodiment, the timing control module integrating the register is further externally equipped with or integrates an external memory. The external memory is electrically connected to the register, and the register reads the external memory to obtain the operation code.

In an embodiment, the image sensor transmits an image signal and the respective clock cycles of the light sources to the image processor by means of a transmission port, and the transmission port is a mobile industry processor interface.

In an embodiment, the image sensor sets a corresponding exposure parameter according to different light sources required in each frame.

In an embodiment, the exposure parameter includes a shutter time and a gain value.

In an embodiment, the light source device emits fluorescence excitation light to a part to be measured, so that the part to be measured is subjected to a fluorescent reaction. The fluorescent reaction includes fluorescent light rays with a specific wavelength. An optical filter receives the fluorescent reaction and filters out a waveband signal with the fluorescence excitation light in the fluorescent reaction to obtain the fluorescent light rays with the specific wavelength.

In conclusion, according to the image sensor device and the method for image and light source synchronization thereof, and the endoscope device in the present disclosure, when the light source device is controlled to start working and illuminates the part to be measured with a set light source, the image sensor is controlled to start working synchronously, thus achieving the effect of preventing the image pictures generated under different light sources from displacing in the switching process. In addition, in a non-synchronous processing mode that images are shotted after the light sources are switched, when the image pictures generated under different light sources are displayed on screens, a plurality of image pictures cannot be shown synchronously in human eyes, that is, in the same period of time, the physician can only watch the image pictures generated by one light source. However, in the present disclosure, when the generated image pictures are displayed on the corresponding screens respectively, due to the persistence of human vision, it looks like continuous pictures when the physician watches the image pictures on the screens, therefore, in the same period of time, the physician can watch the image pictures generated by different light sources respectively, and thus the effect of improving the detection and analysis accuracy of the physician for whether the part to be measured has symptoms or not is achieved.

### BRIEF DESCRIPTION OF THE DRAWINGS

FIG. 1 is a block diagram of Embodiment 1 of an image sensor device according to the present disclosure.

FIG. 2 is a timing diagram of Embodiment 1 of an image sensor device according to the present disclosure.

FIG. 3 is a timing diagram of Embodiment 2 of an image sensor device according to the present disclosure.

FIG. 4 is a timing diagram of Embodiment 3 of an image sensor device according to the present disclosure.

FIG. 5 is a timing diagram of Embodiment 4 of an image sensor device according to the present disclosure.

FIG. 6 is a block diagram of Embodiment 2 of an image sensor device according to the present disclosure.

FIG. 7 is a block diagram of Embodiment 3 of an image sensor device according to the present disclosure.

FIG. 8 is a block diagram of Embodiment 4 of an image sensor device according to the present disclosure.

FIG. 9 is a partial enlarged view of an endoscope device according to the present disclosure.

FIG. 10 is a flowchart of steps of a method for image and light source synchronization of an image sensor device according to the present disclosure.

### DETAILED DESCRIPTION OF THE INVENTION

To fully understand the purpose, characteristics and effect of the present disclosure, the present disclosure is described in detail by means of the following specific embodiments in cooperation with the accompanying drawing as follows:

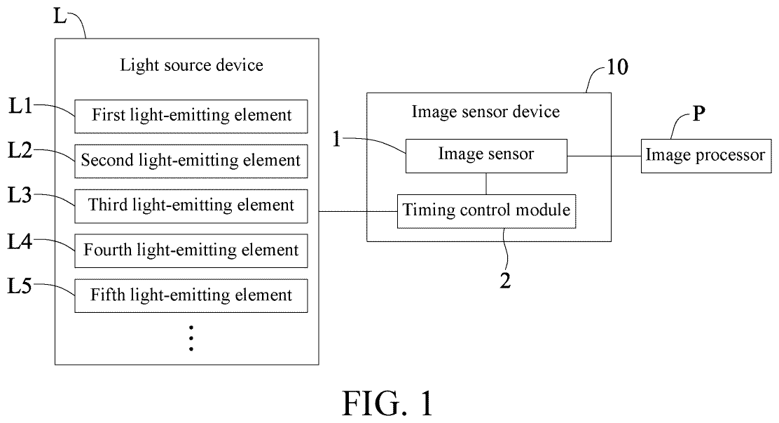

Referring to FIG. 1, which is a block diagram of Embodiment 1 of an image sensor device according to the present disclosure. The image sensor device is suitable for coupling with an image processor P and a light source device L. The light source device L is configured to emit at least two kinds of light sources to illuminate a part to be measured. The image sensor device 10 includes an image sensor 1 and a timing control module 2. The image sensor 1 is electrically connected to the image processor P, and the timing control module 2 is electrically connected to the image sensor 1. The part to be measured is a location of a suspected symptom in a human body, for example, in the esophagus or the neck, but is not limited thereto.

In a specific embodiment, the image processor P may have image processing functions such as color correction, conversion and gamma correction and is configured to output an image information frame. In this embodiment, the image information frame may contain information such as images and light sources.

In a specific embodiment, the light source device L may be a combination of a halogen lamp and a plurality of optical filters or a plurality of light emitting diodes (LEDs) to emit light sources of different colors.

Preferably, the light sources emitted by the light source device L may be at least two of white light, structured light, infrared light, ultraviolet light, fluorescence excitation light and narrow band imaging (NBI) light, which can achieve the effect of providing image pictures under illumination by different light sources. For example, the white light may be generated by a white LED, and the NBI light may be generated by a blue LED, a green LED or a combination thereof.

In a specific embodiment, the image sensor 1 is configured to sense reflected light generated by the part to be measured when illuminated by the light source device L, convert a light signal corresponding to the reflected light into an image signal and then transmit the image signal to the image processor P.

Preferably, the image sensor 1 can transmit the image signal and respective clock cycles of the light sources to the image processor P by means of a transmission port. The transmission port preferably may be a mobile industry processor interface (MIPI), which can achieve the effect of making the image processor acquire the information of the light sources switched by the light source device; and the image sensor 1 preferably may be a complementary metal oxide semiconductor (CMOS).

Preferably, the image sensor 1 can set a corresponding exposure parameter according to different light sources required in each frame, such as a shutter time and a gain value. For example, when the light source device L switches the light source into white light, the image sensor 1 may synchronously set the shutter time and gain value relative to the white light.

In a specific embodiment, the timing control module 2 preferably may be a microcontroller unit (MCU). Specifically, the timing control module 2 synchronously controls the light source device L and the image sensor 1 by using a synchronization signal (Frame Synchronization Pulse, F_sync), and synchronously controls the light source device L to switch and activate between the light sources according to different light sources required by the image sensor 1 in each frame, so that the image sensor 1 generates different image signals corresponding to different light sources.

Preferably, the switching time of the light sources may be less than the time of persistence of human vision (such as 1/16 sec). It is to be noted that the timing control module 2 preferably controls the light source device L with a switch controller (such as an Open Collector) to switch the light sources, so that the effect of reducing the energy consumption of the image sensor device can be achieved; and since the timing control module 2 is integrated into the image sensor device 10, when the image sensor device 10 is arranged in the endoscope device, particularly a disposable endoscope device, arrangement of transmission lines may be reduced to reduce the space used, and the effect of simplifying the circuit arrangement is achieved.

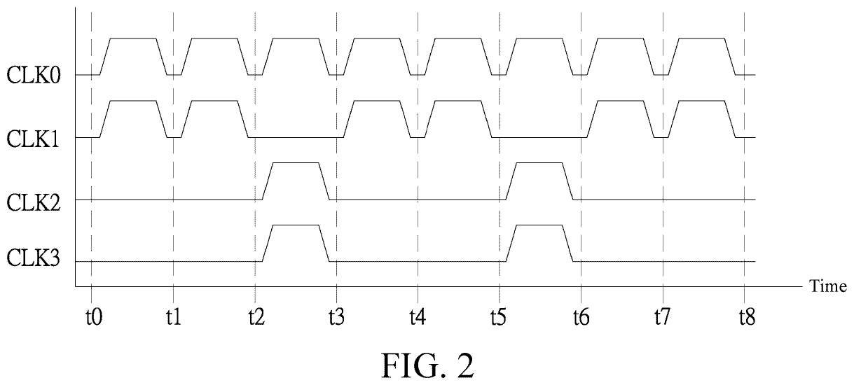

Referring to FIG. 2, which is a timing diagram of Embodiment 1 of an image sensor device according to the present disclosure, where a 0^{th} frame is in a period from t0 to t1, a 1^{st} frame is in a period from t1 to t2, a 2^{nd} frame is in a period from t2 to t3, a 3^{rd} frame is in a period from t3 to t4, a 4th frame is in a period from t4 to t5, a 5th frame is in a period from t5 to t6, a 6th frame is in a period from t6 to t7, and a 7th frame is in a period from t7 to t8.

In this embodiment, the synchronization signal (timing signal CLK0) controls the image sensor 1 to maintain to continuously work at a high level in the periods from t0 to t8 and the like, controls the light source device L to maintain at a high level in the periods from t0 to t2, from t3 to t5, from t6 to t8 and the like and maintain at a low level in the rest periods through a first light-emitting element L1 (timing signal CLK1) that emits white light, and controls the light source device L to maintain at a high level in the periods from t2 to t3, from t5 to t6 and the like and maintain at a low level in the rest periods through a second light-emitting element L2 (timing signal CLK2) that emits structured light; or controls the light source device L to maintain at a high level in the periods from t2 to t3, from t5 to t6 and the like and maintain at a low level in the rest periods through a third light-emitting element L3 (timing signal CLK3) that emits NBI light. Therefore, reflected light sensed by the image sensor 1 in the periods from t0 to t2, from t3 to t5, from t6 to t8 and the like is generated by illuminating the part to be measured with white light, and reflected light sensed by the image sensor 1 in the periods from t2 to t3, from t5 to t6 and the like is generated by illuminating the part to be measured with structured light or NBI light, so that image signals corresponding to different light sources such as white light, white light, structured light, or NBI light, white light, white light, structured light, or NBI light, white light and white light are generated in sequence.

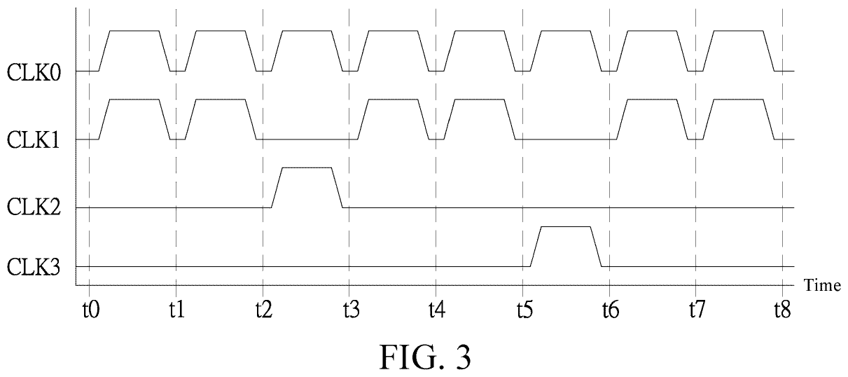

Referring to FIG. 3, which is a timing diagram of Embodiment 2 of an image sensor device according to the present disclosure, where a 0^{th} frame is in a period from t0 to t1, a 1^{st} frame is in a period from t1 to t2, a 2^{nd} frame is in a period from t2 to t3, a 3^{rd} frame is in a period from t3 to t4, a 4th frame is in a period from t4 to t5, a 5th frame is in a period from t5 to t6, a 6th frame is in a period from t6 to t7, and a 7th frame is in a period from t7 to t8.

In this embodiment, the synchronization signal (timing signal CLK0) controls the image sensor 1 to maintain to continuously work at a high level in the periods from t0 to t8 and the like, controls the light source device L to maintain at a high level in the periods from t0 to t2, from t3 to t5, from t6 to t8 and the like and maintain at a low level in the rest periods through the first light-emitting element L1 (timing signal CLK1) that emits white light, controls the light source device L to maintain at a high level in the period from t2 to t3 and maintain at a low level in the rest periods through the second light-emitting element L2 (timing signal CLK2) that emits structured light, and controls the light source device L to maintain at a high level in the period from t5 to t6 and maintain at a low level in the rest periods through the third light-emitting element L3 (timing signal CLK3) that emits NBI light. Therefore, reflected light sensed by the image sensor 1 in the periods from t0 to t2, from t3 to t5, and from t6 to t8 is generated by illuminating the part to be measured with white light, reflected light sensed by the image sensor 1 in the period from t2 to t3 is generated by illuminating the part to be measured with structured light, and reflected light sensed by the image sensor 1 in the period from t5 to t6 is generated by illuminating the part to be measured with NBI light, so that image signals corresponding to different light sources such as white light, white light, structured light, white light, white light, NBI light, white light and white light are generated in sequence. In this embodiment, it is preferably applied to 30 fps pictures, and meets most of existing screen specifications; and moreover, smooth and low-latency display is realized, and meanwhile low power consumption is kept.

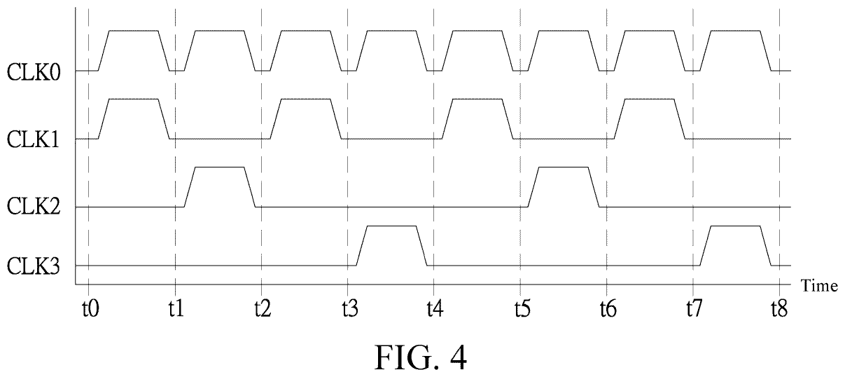

Referring to FIG. 4, which is a timing diagram of Embodiment 3 of an image sensor device according to the present disclosure, where a 0^{th} frame is in a period from t0 to t1, a 1^{st} frame is in a period from t1 to t2, a 2^{nd} frame is in a period from t2 to t3, a 3^{rd} frame is in a period from t3 to t4, a 4th frame is in a period from t4 to t5, a 5th frame is in a period from t5 to t6, a 6th frame is in a period from t6 to t7, and a 7th frame is in a period from t7 to t8.

In this embodiment, the synchronization signal (timing signal CLK0) controls the image sensor 1 to maintain to continuously work at a high level in the periods from t0 to t8 and the like, controls the light source device L to maintain at a high level in the periods from t0 to t1, from t2 to t3, from t4 to t5, from t6 to t7 and the like and maintain at a low level in the rest periods through the first light-emitting element L1 (timing signal CLK1) that emits white light, controls the light source device L to maintain at a high level in the periods from t1 to t2, from t5 to t6 and the like and maintain at a low level in the rest periods through the second light-emitting element L2 (timing signal CLK2) that emits structured light, and controls the light source device L to maintain at a high level in the periods from t3 to t4, from t7 to t8 and the like and maintain at a low level in the rest periods through the third light-emitting element L3 (timing signal CLK3) that emits NBI light. Therefore, reflected light sensed by the image sensor 1 in the periods from t0 to t1, from t2 to t3, from t4 to t5, from t6 to t7 and the like is generated by illuminating the part to be measured with white light, reflected light sensed by the image sensor 1 in the periods from t1 to t2, from t5 to t6 and the like is generated by illuminating the part to be measured with structured light, and reflected light sensed by the image sensor 1 in the periods from t3 to t4, from t7 to t8 and the like is generated by illuminating the part to be measured with NBI light, so that image signals corresponding to different light sources such as white light, structured light, white light, NBI light, white light, structured light, white light and NBI light are generated in sequence. In this embodiment, it is preferably applied to 60 fps pictures, smoother pictures may be displayed on the screen at the same time, and the problem of picture jamming when the physician view images such as white light images, structured light images and NBI light images through the screen is avoided.

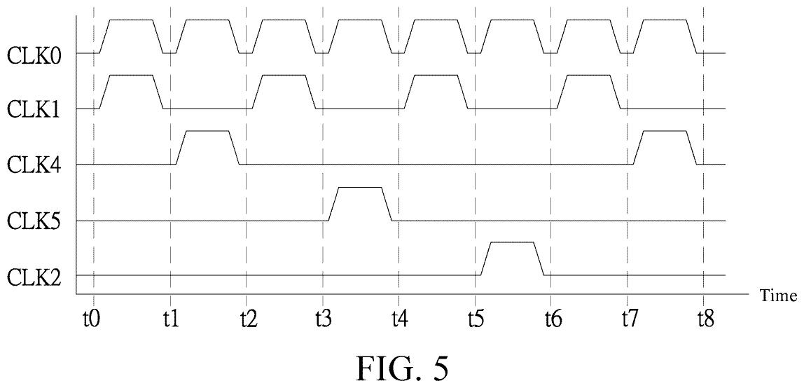

Referring to FIG. 5, which is a timing diagram of Embodiment 4 of an image sensor device according to the present disclosure, where a 0^{th} frame is in a period from t0 to t1, a 1^{st} frame is in a period from t1 to t2, a 2^{nd} frame is in a period from t2 to t3, a 3^{rd} frame is in a period from t3 to t4, a 4th frame is in a period from t4 to t5, a 5th frame is in a period from t5 to t6, a 6th frame is in a period from t6 to t7, and a 7th frame is in a period from t7 to t8.

In this embodiment, the synchronization signal (timing signal CLK0) controls the image sensor 1 to maintain to continuously work at a high level in the periods from t0 to t8 and the like, controls the light source device L to maintain at a high level in the periods from t0 to t1, from t2 to t3, from t4 to t5, from t6 to t7 and the like and maintain at a low level in the rest periods through the first light-emitting element L1 (timing signal CLK1) that emits white light, controls the light source device L to maintain at a high level in the periods from t1 to t2, from t7 to t8 and the like and maintain at a low level in the rest periods through a fourth light-emitting element L4 (timing signal CLK4) that emits blue light, controls the light source device L to maintain at a high level in the period from t3 to t4 and maintain at a low level in the rest periods through a fifth light-emitting element L5 (timing signal CLK5) that emits green light, and controls the light source device L to maintain at a high level in the period from t5 to t6 and maintain at a low level in the rest periods through the second light-emitting element L2 (timing signal CLK2) that emits structured light. Therefore, reflected light sensed by the image sensor 1 in the periods from t0 to t1, from t2 to t3, from t4 to t5, from t6 to t7 and the like is generated by illuminating the part to be measured with white light, reflected light sensed by the image sensor 1 in the periods from t1 to t2, from t7 to t8 and the like is generated by illuminating the part to be measured with blue light, reflected light sensed by the image sensor 1 in the period from t3 to t4 is generated by illuminating the part to be measured with green light, and reflected light sensed by the image sensor 1 in the period from t5 to t6 is generated by illuminating the part to be measured with structured light, so that image signals corresponding to different light sources such as white light, blue light, white light, green light, white light, structured light, white light and blue light are generated in sequence. In this embodiment, it is preferably applied to 120 fps pictures, finer pictures may be displayed on the screen at the same time, and the physician can more clearly see whether the part to be measured has a symptom.

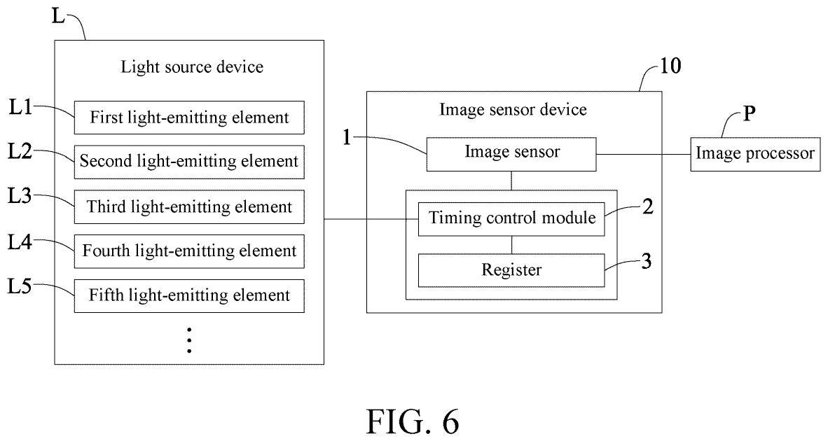

Referring to FIG. 6, which is a block diagram of Embodiment 2 of an image sensor device according to the present disclosure. The timing control module 2 may integrate a register 3 for storing an operation code, and the operation code is configured for setting respective clock cycles of the light sources emitted by the light source device L. Therefore, the light source device may be directly controlled by the image sensor device, and the effect of decreasing the number of signal lines for controlling the light source device is achieved.

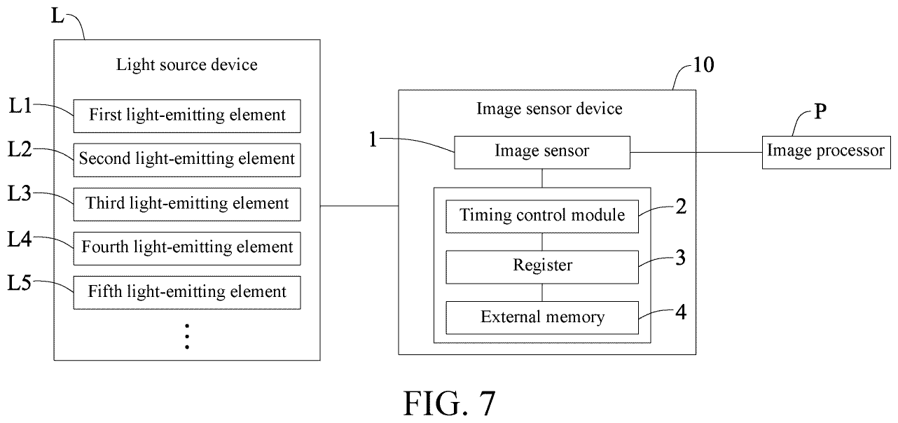

Referring to FIG. 7, which is a block diagram of Embodiment 3 of an image sensor device according to the present disclosure. The timing control module 2 integrating the register 3 may further be externally equipped with or integrates an external memory 4 (for example, an Erasable Programmable Read Only Memory (EPROM)). The external memory 4 is electrically connected to the register 3, and the register 3 reads the external memory 4 to obtain the operation code. Therefore, when the image sensor device 10 is arranged in an endoscope device, especially a disposable endoscope device, an additional circuit does not need to be externally installed, the space used is reduced, and the effect of reducing the circuit complexity is achieved.

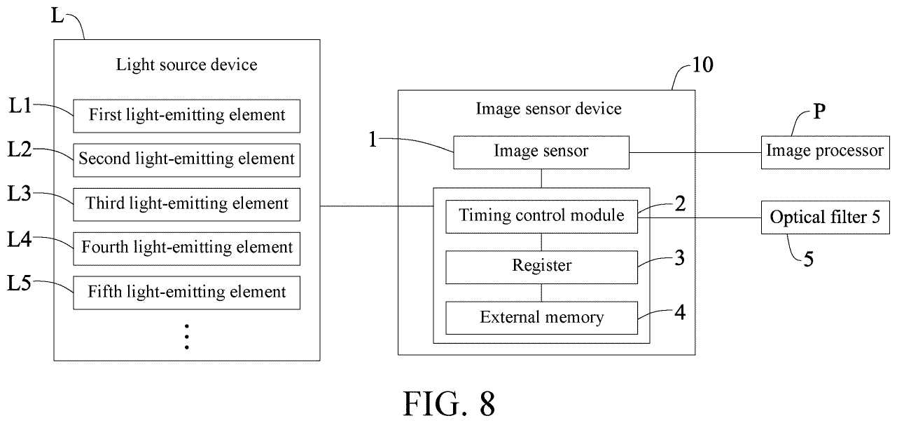

Referring to FIG. 8, which is a block diagram of Embodiment 4 of an image sensor device according to the present disclosure. Compared with Embodiment 1, the image sensor device may further include an optical filter 5, which is arranged in front of the image sensor 1 to filter out light rays with certain wavelengths. Specifically, the light source device L may emit fluorescence excitation light towards the part to be measured, so that the part to be measured is subjected to a fluorescence reaction. The fluorescence reaction includes fluorescence light rays with a specific wavelength. The optical filter 5 receives the fluorescence reaction and filters out a waveband signal with the fluorescence excitation light in the fluorescence reaction to obtain the fluorescence light rays with the specific wavelength.

For example, in this embodiment, an Indocyanine green (ICG) developing function may be provided. Specifically, the ICG is a non-radioactive fluorescent dye, of which the main absorption wavelength is about 720-810 nm, and the reaction wavelength of the main fluorescence emitted after absorption is about 810-900 nm, so that the optical filter 5 that can filter out at least the light rays with the wavelength of 700-810 nm is selected; and the light source device L emits the fluorescence excitation light with the central wavelength of 750-810 nm towards the part to be measured, and thus the image sensor 1 can convert the fluorescence light rays with the wavelength of about 815-880 nm into corresponding image signals.

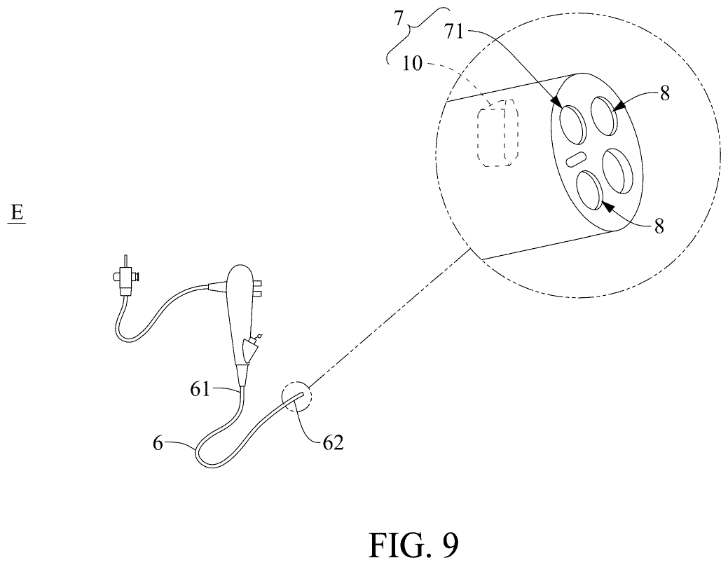

Referring to FIG. 9, which is a partial enlarged view of a specific embodiment of an endoscope device according to the present disclosure. The endoscope device E is suitable for combining with the light source device L described above, and the endoscope device E includes an extension tube 6, an imaging assembly 7 and a light guide member 8. The imaging assembly 7 and the light guide member 8 are arranged on the extension tube 6 respectively.

In a specific embodiment, the extension tube 6 is provided with a proximal end 61 and a distal end 62 opposite to the proximal end 61. The proximal end 61 of the extension tube 6 may be connected to an operating handle, and the distal end 62 of the extension tube 6 may extend into the body of a patient.

In a specific embodiment, the imaging assembly 7 includes a lens group 71 and the image sensor device 10 described above. The lens group 71 is located close to the distal end 62 of the extension tube 6. The image sensor device 10 is positioned in the extension tube 6 and is clamped to the lens group 71.

In a specific embodiment, the light guide member 8 is located close to the distal end 62 of the extension tube 6 and is beside the lens group 71, so as to guide the light source emitted by the light source device L into the body of the patient, and for example, but not limited, the light guide member 8 may be an optical fiber bundle.

Preferably, the endoscope device according to the present disclosure may further include the optical filter 5, which is arranged in front of the image sensor device 10 to filter out light rays with certain wavelengths.

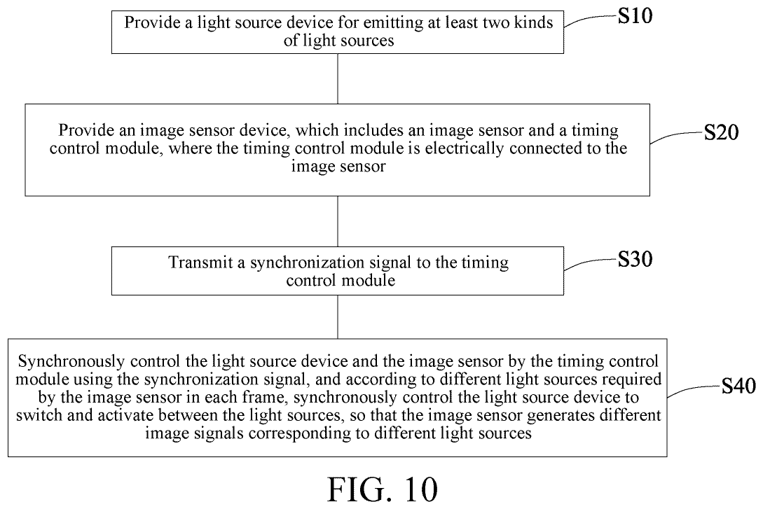

Referring to FIG. 10, which is a flowchart of steps of a method of image and light source synchronization of an image sensor device according to the present disclosure. The method includes the following steps:

The light source device L is provided for emitting at least two kinds of light sources (step S10). Preferably, the light sources emitted by the light source device L to a part to be measured may be at least two of white light, structured light, infrared light, ultraviolet light, fluorescence excitation light and NBI light respectively. For example, the white light may be generated by a white LED, and the NBI light may be generated by a blue LED, a green LED or a combination thereof. The part to be measured is a location of a suspected symptom in a human body, for example, in the esophagus or the neck, but is not limited thereto.

An image sensor device 10 is provided, which includes the image sensor 1 and the timing control module 2, where the timing control module 2 is electrically connected to the image sensor 1 (step S20). The image sensor 1 may transmit an image signal and respective clock cycles of the light sources to the image processor P by means of the transmission port.

A synchronization signal is transmitted to the timing control module 2 (step S30).

The light source device L and the image sensor 1 are synchronously controlled by the timing control module 2 using the synchronization signal, and according to different light sources required by the image sensor 1 in each frame, the light source device L is synchronously controlled to switch and activate between the light sources, so that the image sensor 1 generates different image signals corresponding to different light sources (step S40).

Specifically, the image sensor 1 can be configured to sense reflected light generated by the part to be measured when illuminated by the light source device L, convert the light signal corresponding to the reflected light into the image signal and transmit the image signal to the image processor P. Preferably, the image sensor 1 may be a complementary metal oxide semiconductor.

Preferably, the image sensor 1 can set a corresponding exposure parameter according to different light sources required in each frame, and the exposure parameter may include a shutter time and a gain value.

Preferably, the timing control module 2 may integrate the register 3 to store an operation code, and the operation code is configured for setting respective clock cycles of the light sources emitted by the light source device L. Furthermore, the timing control module 2 integrating the register 3 may further be externally equipped with or integrate the external memory 4. The external memory 4 is electrically connected to the register 3, and the register 3 reads the external memory 4 to obtain the operation code.

Preferably, the light rays with certain wavelengths can be filtered out by the optical filter 5. Specifically, the light source device L may emit fluorescence excitation light towards the part to be measured, so that the part to be measured is subjected to a fluorescence reaction. The fluorescence reaction includes fluorescence light rays with a specific wavelength. The optical filter 5 receives the fluorescence reaction and filters out a waveband signal with the fluorescence excitation light in the fluorescence reaction to obtain the fluorescence light rays with the specific wavelength.

In conclusion, according to the image sensor device and the method for image and light source synchronization thereof, and the endoscope device in the present disclosure, when the light source device is controlled to start working and illuminates the part to be measured with a set light source, the image sensor is controlled to start working synchronously, thus achieving the effect of preventing the image pictures generated under different light sources from displacing in the switching process. In addition, in a non-synchronous processing mode that images are shotted after the light sources are switched, when the image pictures generated under different light sources are displayed on screens, a plurality of image pictures cannot be shown synchronously in human eyes, that is, in the same period of time, the physician can only watch the image pictures generated by one light source. However, in the present disclosure, when the generated image pictures are displayed on the corresponding screens respectively, due to the persistence of human vision, it looks like continuous pictures when the physician watches the image pictures on the screens, therefore, in the same period of time, the physician can watch the image pictures generated by different light sources respectively, and thus the effect of improving the detection and analysis accuracy of the physician for whether the part to be measured has symptoms or not is achieved.

The present disclosure has been disclosed in the above with preferred embodiments, but those skilled in the art should understand that the embodiments are only used for describing the present disclosure and should not be construed as limiting the scope of the present disclosure. It is to be noted that all changes and substitutions equivalent to the embodiments should fall within the scope of the present disclosure. Therefore, the scope of protection of the present disclosure shall be subject to the definition of the claims.

While the present disclosure has been described by means of specific embodiments, numerous modifications and variations could be made thereto by those skilled in the art without departing from the scope and spirit of the present disclosure set forth in the claims.

What is claimed is:

1. An image sensor device, suitable for coupling with an image processor and a light source device, wherein the light source device is configured to emit at least two kinds of light sources to illuminate a part to be measured, and the image sensor device comprises:
an image sensor, electrically connected to the image processor and configured to sense reflected light generated by the part to be measured when illuminated by the light source device, and convert a light signal corresponding to the reflected light into an image signal and then transmit the image signal to the image processor; and
a timing control module, electrically connected to the image sensor and configured to use a synchronization signal to synchronously control the light source device and the image sensor, and according to different light sources required by the image sensor in each frame, synchronously control the light source device to switch and activate between the light sources, so that the image sensor generates different image signals corresponding to different light sources.

2. The image sensor device according to claim 1, wherein the light sources emitted by the light source device are at least two of white light, structured light, infrared light, ultraviolet light, fluorescence excitation light and narrow band imaging light.

3. The image sensor device according to claim 1, wherein the timing control module integrates a register for storing an operation code, and the operation code is configured for setting respective clock cycles of the light sources emitted by the light source device.

4. The image sensor device according to claim 3, wherein the timing control module integrating the register is further externally equipped with or integrates an external memory, the external memory is electrically connected to the register, and the register reads the external memory to obtain the operation code.

5. The image sensor device according to claim 3, wherein the image sensor transmits the image signal and the respective clock cycles of the light sources to the image processor by means of a transmission port, and the transmission port is a mobile industry processor interface.

6. The image sensor device according to claim 1, wherein the image sensor sets a corresponding exposure parameter according to different light sources required in each frame.

7. The image sensor device according to claim 6, wherein the exposure parameter comprises a shutter time and a gain value.

8. The image sensor device according to claim 1, further comprising an optical filter, arranged in front of the image sensor to filter out light rays with certain wavelengths.

9. An endoscope device, suitable for combining with a light source device, and the endoscope device comprising:
an extension tube, provided with a proximal end and a distal end opposite to the proximal end;
an imaging assembly, comprising a lens group located close to the distal end of the extension tube, and the image sensor device according to claim 1 positioned in the extension tube and clamped to the lens group; and
a light guide member, located close to the distal end of the extension tube and being beside the lens group,
wherein the light source device is suitable for being located in the extension tube and close to the light guide member.

10. The endoscope device according to claim 9, further comprising an optical filter, arranged in front of the image sensor device.

11. A method for image and light source synchronization of an image sensor device, comprising the following steps:
providing a light source device for emitting at least two kinds of light sources;
providing an image sensor device, wherein the image sensor device comprises an image sensor and a timing control module, and the timing control module is electrically connected to the image sensor;
transmitting a synchronization signal to the timing control module; and
synchronously controlling the light source device and the image sensor by the timing control module using the synchronization signal, and according to different light sources required by the image sensor in each frame, synchronously controlling the light source device to switch and activate between the light sources, so that the image sensor generates different image signals corresponding to different light sources.

12. The method for image and light source synchronization of the image sensor device according to claim 11, wherein the light sources emitted by the light source device are at least two of white light, structured light, infrared light, ultraviolet light, fluorescence excitation light and narrow band imaging light.

13. The method for image and light source synchronization of the image sensor device according to claim 11, wherein the timing control module integrates a register for storing an operation code, and the operation code is configured for setting respective clock cycles of the light sources emitted by the light source device.

14. The method for image and light source synchronization of the image sensor device according to claim 13, wherein the timing control module integrating the register is further externally equipped with or integrates an external memory, the external memory is electrically connected to the register, and the register reads the external memory to obtain the operation code.

15. The method for image and light source synchronization of the image sensor device according to claim 13, wherein the image sensor transmits an image signal and the respective clock cycles of the light sources to the image processor by means of a transmission port, and the transmission port is a mobile industry processor interface.

16. The method for image and light source synchronization of the image sensor device according to claim 11, wherein the image sensor sets a corresponding exposure parameter according to different light sources required in each frame.

17. The method for image and light source synchronization of the image sensor device according to claim 16, wherein the exposure parameter comprises a shutter time and a gain value.

18. The method for image and light source synchronization of the image sensor device according to claim 11, wherein the light source device emits fluorescence excitation light to a part to be measured, so that the part to be measured is subjected to a fluorescent reaction, the fluorescent reaction comprises fluorescent light rays with a specific wavelength, and an optical filter receives the fluorescent reaction and filters out a waveband signal with the fluorescence excitation light in the fluorescent reaction to obtain the fluorescent light rays with the specific wavelength.
